(19) United States
(12) Patent Application Publication
(10) Pub. No.: US 2008/0166292 A1
(43) Pub. Date: Jul. 10, 2008

(54) Pharmaceutical Dosing Method

(75) Inventors: Paul D. Levin, Pittsburgh, PA (US); Gerald S. Orban, Gibsonia, PA (US)

Correspondence Address: GREGORY L BRADLEY;MEDRAD INC, ONE MEDRAD DRIVE, INDIANOLA, PA, 15051, US

(73) Assignee: MEDRAD, INC., Indianola, PA (US)

(21) Appl. No.: 11/967,422

(22) Filed: Dec. 31, 2007

Related U.S. Application Data
(60) Provisional application No. 60/878,333, filed on Jan. 1, 2007.

(51) Int. Cl. A61K 51/00; A61B 6/00

(52) U.S. Cl. 424/1.11; 600/431

(57) ABSTRACT

Systems, devices, and methods for more accurately determining a radiopharmaceutical dose administered to a patient by relying on a time factor. Particularly, broadly contemplated herein is the administration of a dose on the basis of an elapsed time from when a dose was last accurately measured in the past to when it is injected into the patient. As such, when a dose is first measured, that timepoint is preferably recorded whereupon the time of injection or administration into a patient is also recorded. Based on the original measured dose, the radionuclide (and thus its known decay rate) and the time elapsed, the dose is calculated and not directly measured on injection. The clocks on the filling station and the transport cart are synchronized to each other or to a known and accepted time standard. In this manner, there is temporal continuity and no inaccuracies of time or loss of time occurs between measurement and injection.

### FIELD OF THE INVENTION

The present invention relates to delivery methods, systems and components thereof for use with hazardous or toxic pharmaceutical substances, and more particularly to the provision of accurate doses of such substances.

### BACKGROUND OF THE INVENTION

As used herein, the term “pharmaceutical” refers to any substance to be injected or otherwise delivered into the body (either human or animal) in a medical procedure and includes, but is not limited, substances used in imaging procedures (for example, contrast media) and therapeutic substances. A number of such pharmaceutical substances pose a danger to both the patient and the personnel administering the substance if not handled and/or injected properly. Examples of hazardous pharmaceuticals include, but are not limited to, radiopharmaceuticals, biological pharmaceuticals, chemotherapeutic pharmaceuticals and gene therapeutic pharmaceuticals.

Examples of use of a radiopharmaceutical include positron emission tomography (PET) and single-photon emission computerized tomography (SPECT), which are noninvasive, three-dimensional, imaging procedures that provide information regarding physiological and biochemical processes in patients. The first step in producing PET images or SPECT images of, for example, the brain or another organ, is to inject the patient with a dose of the radiopharmaceutical. The radiopharmaceutical is generally a radioactive substance that can be absorbed by certain cells in the brain or other organ, concentrating it there. For example, fluorodeoxyglucose (FDG) is a normal molecule of glucose, the basic energy fuel of cells, to which is attached a radionuclide or radioactive fluor. The radionuclide is produced in a cyclotron equipped with a unit to synthesize the FDG molecule.

Cells (for example, in the brain), which are more active in a given period of time after an injection of FDG, will absorb more FDG because they have a higher metabolism and require more energy. The radionuclide in the FDG molecule suffers a radioactive decay, emitting a positron. When a positron collides with an electron, an annihilation occurs, liberating a burst of energy in the form of two beams of gamma rays in opposite directions. The PET scanner detects the emitted gamma rays to compile a three dimensional image.

In that regard, after injecting the radiopharmaceutical, the patient is typically placed on a moveable bed that slides by remote control into a circular opening of the scanner referred to as the gantry. Positioned around the opening, and inside the gantry, are several rings of radiation detectors. Each detector emits a brief pulse of light every time it is struck with a gamma ray coming from the radionuclide within the patient's body. The pulse of light is amplified, by a photomultiplier, and the information is sent to the computer that controls the apparatus.

The timing of injection is very important. After the generation of the radiopharmaceutical, a countdown begins. After a certain time, which is a function of the half-life of the radionuclide, the radiation level of the radiopharmaceutical dose falls exactly to a level required for the measurement by the scanner. In conventional practice, the radiation level of the radiopharmaceutical volume or dose is typically measured using a dose calibrator. Using the half-life of the radionuclide, the time that the dose should be injected to provide the desired level of radioactivity to the body is calculated. When that time is reached, the radiopharmaceutical dose is injected using a manually operated syringe.

Most PET radionuclides have short half-lives. Under proper injection procedures, these radionuclides can be safely administered to a patient in the form a labeled substrate, ligand, drug, antibody, neurotransmitter or other compound normally processed or used by the body (for example, glucose) that acts as a tracer of specific physiological and biological processes.

Excessive radiation to technologists and other personnel working in the scanner room can pose a significant risk, however. Although the half-life of the radiopharmaceutical is rather short and the applied dosages are themselves not harmful to the patient, administering personnel are exposed each time they work with the radiopharmaceuticals and other contaminated materials under current procedures. Constant and repeated exposure over an extended period of time can be harmful.

A number of techniques used to reduce exposure include minimizing the time of exposure of personnel, maintaining distance between personnel and the source of radiation and shielding personnel from the source of radiation. In general, the radiopharmaceuticals are typically delivered to a nuclear medicine facility from another facility equipped with a cyclotron in, for example, a lead-shielded container. Often, the radiopharmaceutical is manually drawn from such containers into a shielded syringe. See, for example, U.S. Pat. No. 5,927,351 disclosing a drawing station for handling radiopharmaceuticals for use in syringes. Remote injection mechanisms can also be used to maintain distance between the operator and the radiopharmaceutical. See, for example, U.S. Pat. No. 5,514,071, disclosing an apparatus for remotely administering radioactive material from a lead encapsulated syringe.

It has long been recognized as very desirable to develop devices, systems and methods through which toxic or hazardous pharmaceuticals (for example, radiopharmaceuticals) can be administered in controlled manner to enhance their effectiveness and patient safety, while reducing exposure of administering personnel to such hazardous pharmaceuticals.

Conventional systems have also long presented difficulties in their capacity to deliver accurately measured doses of radiopharmaceutical substances arrangements. Typically, a dose is measured directly only immediately prior to injection into or delivery to a patient. At the same time, such measurement is normally carried out by an expensive and bulky calibrator. Due to factors such as radioactive decay, the actual dose delivered often deviates from the initial measurement, while the expense of providing a conventional calibrator is at times prohibitive.

Buck, infra, relates to a feedback arrangement for accurately dosing a patient, particularly in connection with a coil in a metering section. However, it is recognized herein that an even more efficient and effective arrangement for delivering an accurate dose is attainable.

Accordingly, a compelling need has been recognized in connection with the provision of accurately measured doses to a patient, even more cost-efficiently and in even more of a manner to obviate the measurement discrepancies mentioned above.

### SUMMARY OF THE INVENTION

Broadly contemplated herein, in accordance with at least one presently preferred embodiment of the present invention, are systems, devices, and methods for more accurately determining a radiopharmaceutical dose administered to a patient by relying on a time factor. Particularly, broadly contemplated herein is the administration of a dose on the basis of an elapsed time from when a dose was last accurately measured in the past (e.g., when initially dispensed) to when it is injected into the patient.

As such, when a dose is first measured, that timepoint is preferably recorded whereupon the time of injection or administration into a patient is also recorded. Based on the original measured dose, the radionuclide (and thus its known decay rate) and the time elapsed, the dose is calculated and not directly measured on injection. The clocks on the dispensing station and the transport station are synchronized to each other or to a known and accepted time standard. In this manner, there is temporal continuity and no time is lost between measurement and injection.

The novel features which are considered characteristic of the present invention are set forth herebelow. The invention itself, however, both as to its construction and its method of operation, together with additional objects and advantages thereof, will be best understood from the following description of the specific embodiments when read and understood in connection with the accompanying drawings.

### BRIEF DESCRIPTION OF THE DRAWINGS

FIG. 1A illustrates a schematic representation of a system in the context of which embodiments of the present invention may readily be employed.

FIG. 1B illustrates a top cross-sectional view of a shielded container for a fluid delivery set.

FIG. 1C illustrates a side cross-sectional view of another shielded container for a fluid delivery set.

FIG. 2A illustrates a perspective view of an injector and a syringe adapter.

FIG. 2B illustrates a perspective view of injector control units.

FIG. 3 provides a perspective view of a portion of the system of FIG. 1A, wherein the injector head and syringe adapter have been lowered so that the syringe is positioned within the dose calibration unit.

FIG. 4A illustrates a perspective view of the adapter of FIG. 2A detached from the injector with the syringe attached thereto.

FIG. 4B illustrates a perspective view of the adapter of FIG. 2A detached from the injector with the syringe detached therefrom.

FIG. 4C illustrates a side cross-sectional view of a portion of the system of FIGS. 1 through 4B.

FIG. 5A illustrates a side cross-sectional view of an arrangement in which dose calibration is provided by placing a pressurizing device and a source of radiopharmaceutical within a shielded dose calibrator.

FIG. 5B illustrates a side cross-sectional view of an arrangement in which dose calibration is provided by placing a source of radiopharmaceutical within a shielded dose calibrator.

FIG. 5C illustrates a side cross-sectional view of an arrangement in which dose calibration is provided by placing a radiation detector in line between a pressurizing device and a source of radiopharmaceutical within a shielded dose calibrator.

FIG. 5D illustrates a side cross-sectional view of an arrangement in which dose calibration is provided by placing a radiation detector in line with the exit line of a pressurizing device.

FIG. 6 illustrates another example of a conventional fluid delivery system of the present invention in the context of which embodiments of the present invention may readily be employed.

FIG. 7 schematically illustrates a dosing system including a filling station and transport cart with clocks for mutual synchronization.

FIG. 8a schematically illustrates an alternative embodiment of a dosing system including a filling station and transport cart each a clock for mutual synchronization.

FIG. 8b schematically illustrates an alternative embodiment of a dosing system including a filling station and transport cart with clocks for mutual synchronization.

FIG. 8c schematically illustrates an alternative embodiment of a dosing system including a filling station and transport cart with an external clock for mutual synchronization.

### DESCRIPTION OF THE PREFERRED EMBODIMENTS

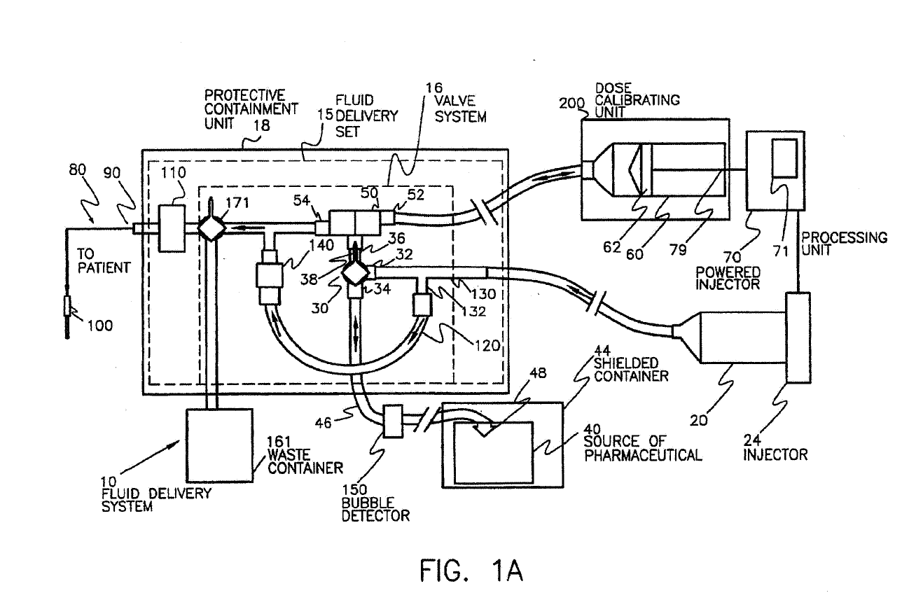

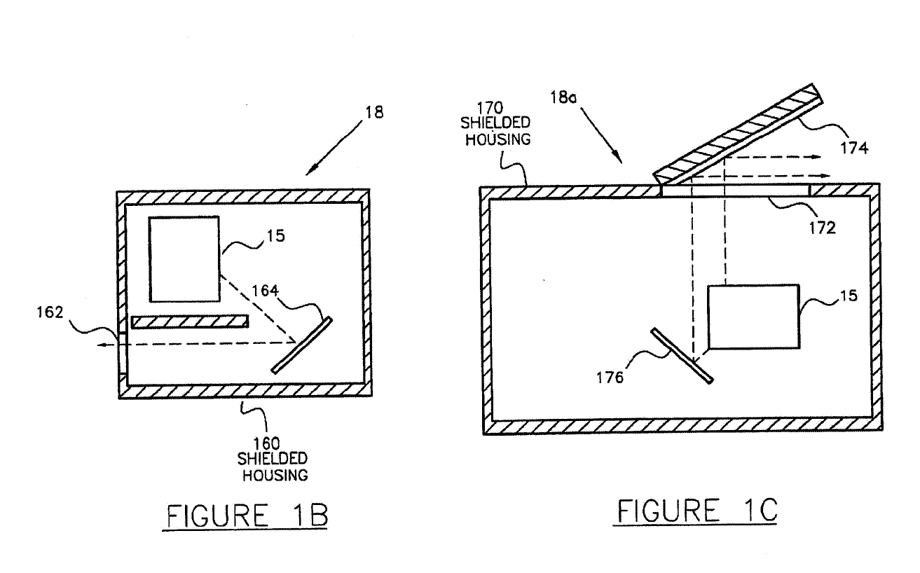

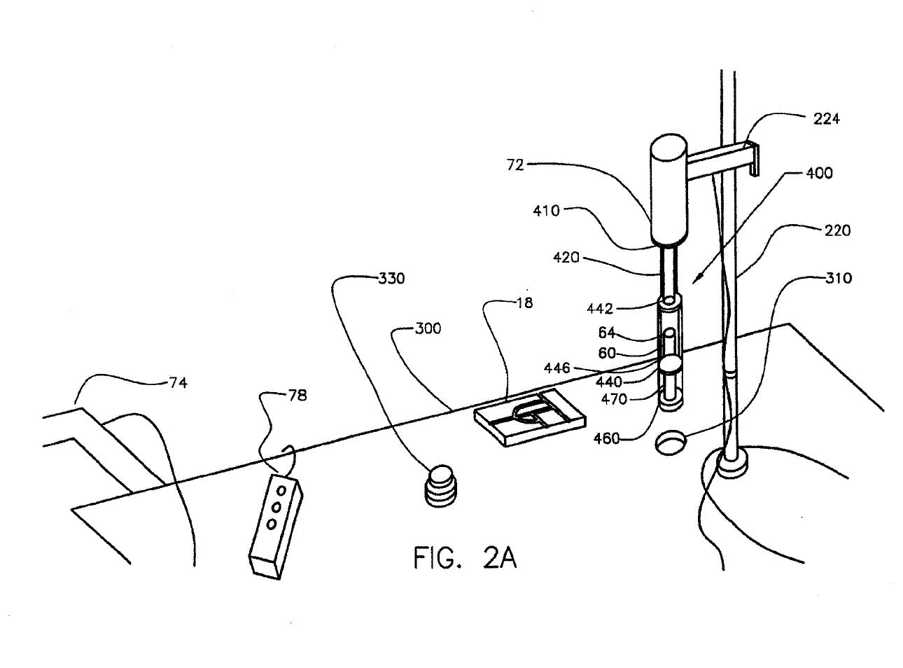

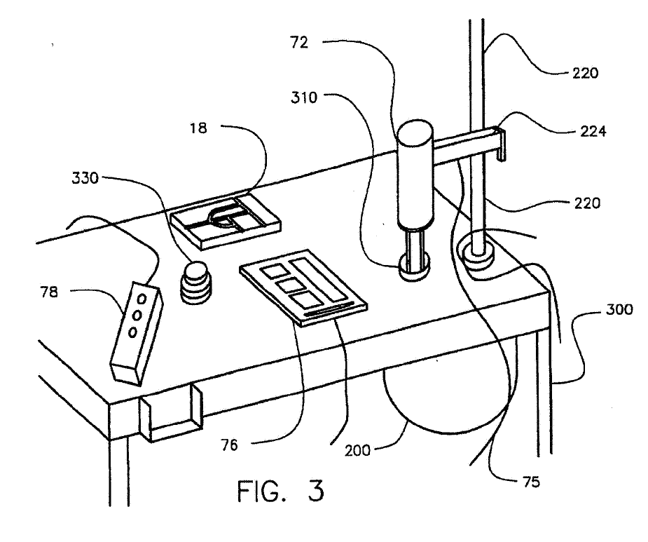

FIGS. 1A-5D illustrate a conventional system for dispensing hazardous pharmaceuticals, as disclosed in U.S. Pat. No. 6,767,319 to Reilly et al. (and assigned to the assignee of the present invention). This patent (the “'319 patent”) is fully incorporated by reference as if set forth in its entirety herein. By way of a set of completely illustrative and non-restrictive examples, the systems broadly contemplated and disclosed in the '319 patent constitute suitable environments in which embodiments of the present invention may be employed.

At the same time, though the embodiments of the present invention may be employed in a wide variety of settings and environments, the '319 patent may be referred to for useful background information for better appreciating the embodiments of the present invention and their manner of functioning. FIGS. 1A-5D, as discussed below and included with the instant application, are taken from the '319 patent.

As illustrated in FIG. 1A, a conventional system 10 includes a fluid delivery set or system 15 including a valve system 16 that provides a fluid connection for a saline source 20 (for example, a syringe), a source 40 of a pharmaceutical to be injected into a patient, a pressurizing chamber or unit for the pharmaceutical (for example, a syringe 60 in fluid connection with a powered injector 70 in the embodiment of FIG. 1) and a fluid path set 80 that is connectable to the patient (via, for example, tubing terminating in a catheter 100). In general, the fluid delivery set 15, valve system 16 and other elements of the present invention enable purging of air from the system, filling of syringe 60 with the pharmaceutical, delivery of the pharmaceutical (for example, injecting the pharmaceutical into the patient) via syringe 60, and providing a saline flush, while minimizing or eliminating exposure of administering or operating personnel to the detrimental effects of the pharmaceutical and minimizing or eliminating creation of contaminated waste. Moreover, fluid delivery set 15 and other elements of the present invention also facilitate safe delivery of the pharmaceutical to multiple destinations (for example, injection into a series patients).

In the system of FIG. 1, valve system 16 includes a three-way stopcock 30 including a first port 32 that is in fluid connection with saline syringe 20. A second port 34 of stopcock 30 is in fluid connection with source 40 of a toxic or hazardous pharmaceutical (for example, a radiopharmaceutical). Source 40 of the pharmaceutical is preferably enclosed within a container 44 that is designed to reduce the risk of contamination of personnel administering the pharmaceutical. For example, in the case of a radiopharmaceutical, the container can be fabricated from lead or tungsten to substantially prevent exposure of such personnel to undesirably high levels of radiation.

A third port 36 of stopcock 30 is in fluid connection with, for example, a dual check valve 50. The flow through stopcock 30 is controlled via control 38. A first port 52 of dual check valve 50 is in fluid connection with syringe 60 that is preferably in operative connection with powered injector 70. A second port 54 of dual check valve 50 is preferably in fluid connection with patient fluid path set 80 that includes, for example, flexible tubing 90 connected to catheter 100. Preferably, patient fluid path set 80 is disposable on a per patient basis to reduce the likelihood of cross-contamination when system 10 is used for injection of fluids into multiple patients. Patient fluid path set 80 is preferably in fluid connection with second port 54 of dual check valve 50 via a one-way check valve 110 to further reduce the likelihood of cross-contamination.

Preferably, saline source 20 is also in fluid connection with fluid path set 80 via bypass tubing or conduit 120 of valve system 16 to provide, for example, flush and KVO (keep vein open) functions on demand without having to adjust control 38 of valve system 16. In the system of FIG. 1, a tee 130 is positioned between saline source 20 and stopcock 30. A side port 132 of tee 130 is in fluid connection with bypass tubing 120. Bypass tubing 120 is preferably in fluid connection with check valve 110 (and thereby with fluid path set 80) via a one-way check valve 140.

In injection procedures and other fluid delivery operations in which non-hazardous pharmaceuticals are delivered, purging air from the entire fluid path (including, the fluid path between a source of the pharmaceutical and the delivery point) typically includes the forcing an amount of the pharmaceutical through the fluid path to, for example, a waste receptor before beginning the procedure (for example, before insertion of a catheter into the patient). However, in the case of a hazardous pharmaceutical such as a radiopharmaceutical, it is very desirable to minimize or eliminate the creation of waste pharmaceutical. Moreover, as discussed above, it is also preferable in the case of a hazardous pharmaceutical to minimize exposure of administering personnel to the pharmaceutical. Systems in accordance with the present invention thus preferably enable purging of air from the entirety of fluid delivery set 15 (and preferably, also from patient fluid path set 80) before connection of fluid delivery set 15 to pharmaceutical source 40. In this manner, exposure of administering personnel to hazardous materials during purging is eliminated and no hazardous waste is generated.

After connecting fluid delivery set 15, which is fluid filled and purged of air, to pharmaceutical source 40, air can be introduced into fluid delivery system 10 from pharmaceutical source 40. Thus, precautions are preferably taken as known in the art to reduce the likelihood of introduction of air into system 10 from pharmaceutical source 40. Moreover, a bubble detector 150 can be placed in communication with line 46 to detect if air is drawn from pharmaceutical source 40. Examples of a bubble detector suitable for use in the present invention include the BDF/BDP series ultrasonic air bubble detectors available from Introtek of Edgewood, N.Y.

In the case that it is desirable to purge system 10 (for example, in the case that air is found in one of the fluid path lines), a waste container 161 (which is preferably shielded) is preferably provided. In the system of FIG. 1A, waste container 161 is in fluid connection with a control valve 171 (similar in operation to control valve 30) which is in line just before check valve 110. Control valve 171 can be controlled remotely or automated to reduce likelihood of exposure of operating personnel to the toxic pharmaceutical. It is also possible, for example, to provide valve 50 with control in a manner known to those skilled in art such that fluid can be purged back to source 40. In general, system 10 is purged using syringe 60 and/or saline source 20 as described below.

During operation of system 10, saline syringe 20 (which can be a hand syringe or a syringe powered by an injector 24) is first filled with saline. Saline syringe 20 is then connected to valve system 16 of fluid delivery set 15 via first port 32 on three-way stopcock 30. Saline syringe 20 is preferably used to purge air from system 10. Saline syringe 20 also provides a flush to patient fluid path set 80 after injection of pharmaceutical(s) to ensure that substantially all the pharmaceutical is injected into the patient and to ensure that very little if any of the toxic or hazardous pharmaceutical remains, for example, within fluid path set 80.

Syringe 60 is attached to injector 70. In the case of injection of a radiopharmaceutical, at least syringe 60 of injector 70 is preferably enclosed within a shielded container during an injection procedure. In one embodiment, the shielded container is a radiation dose calibration unit 200 as discussed in further detail below. Air is first preferably expelled from syringe 60 by advancing plunger 62 of syringe 60 toward syringe tip 64. Syringe 60 is then connected to dual check valve 50 of valve system 16 via first port 52. Patient fluid path set 80 is connected to valve system 16 via one-way check valve 110.

Control 38 is adjusted to place saline syringe 20 in fluid connection with tubing 46. Tubing 46 can, for example, terminate in a spike 48 or other connection member to cooperate with a septum 45 on source 40 (for example, a bottle) as known in the art. A small volume of saline is injected or expelled from saline syringe 20 to purge air from tubing 46 and spike 48. Control 38 is then adjusted to place saline syringe 20 in fluid connection with dual check valve 50. A small volume of saline is expelled to purge flush bypass line 120 of air. Dual check valve 50 provides sufficient resistance to flow such that saline expelled from saline syringe 20 passes through bypass line 120 rather than through dual check valve 50.

Injector 70 is used to retract plunger 62 to draw saline from saline syringe 20. Injector 70 is then used to expel air in line between syringe 60 and catheter 100 by expelling (via advancement of plunger 62) the saline therefrom. At this point, all lines of system 10 are free of air and filled with saline. Syringe 60 is substantially empty except for a small amount of saline not expelled.

At this point, injector syringe 60 is preferably positioned within dose calibrating unit 200 or other radiation containment device in the case of injection of a radiopharmaceutical. Container 44 is opened and pharmaceutical source 40 is spiked to place source 40 in fluid connection with valve system 16. Spiking of pharmaceutical source 40 can be done automatically, remotely or robotically to reduce or prevent exposure of operating personnel. The patient is then connected to patient fluid path set 80 via catheter 100. System 10 is now ready for an injection. The pharmaceutical is drawn into syringe 60 by retraction of plunger 62 relative to syringe tip 64 and then injected into the patient by advancement of plunger 62 relative to syringe tip 64. Saline is then expelled from saline syringe 20, passing through bypass line 120, to flush the pharmaceutical from patient fluid path set 80. All of these functions are accomplished with little on no exposure of the operator or administering personnel to radiation.

In that regard, all adjustments of control 38 were made before the radiopharmaceutical was drawn into fluid delivery set 15. Control 38 can also be adjusted remotely or automatically (for example, via electronic/computer control) in, for example, cases when some pharmaceutical is within fluid delivery set 15 (for example, in a second or subsequent procedure in a case in which fluid delivery set 15 is used for multiple deliveries/injections) to prevent exposure of administering personnel. Other types of valve systems or assemblies, for example, a manifold system, can be used to affect the control of valve assembly 16.

Fluid delivery set 15 is preferably disposable after one or more uses to, for example, reduce the risk of cross-contamination between patients. Fluid delivery set 15, including valve system 16, and/or other components of system 10 can be placed within a protective containment unit 18 during use thereof to further shield personnel from radiation that may emanate from, for example, valve system 16. FIG. 1B illustrates one embodiment of protective containment unit or shielded container 18 for fluid delivery set 15. In general, radioactive rays emanate in straight lines from a radiation source. Containment unit 18 provides a view of fluid delivery set 15 without providing a straight line of sight between the viewer and fluid delivery set 15. In that regard, it is often desirable for administering personnel to have a view of tubing in a fluid path to, for example, provide visual assurance of the absence of air bubbles. Containment unit 18 includes a shielded housing 160 having a view port 162. Radioactive rays cannot escape through view port 162, as there is no line of sight (that is, unobstructed line) between view port 162 and fluid delivery set 15. Containment unit 18 includes a mirrored surface 164 to provide a view of fluid delivery set 15. FIG. 1C illustrates another embodiment of a containment unit 18a in which a view of fluid delivery set 15 is provided by mirrored surface 174, which is in alignment with fluid delivery set 15 via view port 172. One or more additional mirrored surfaces 176 can be provided to give further views of fluid delivery set 15.

In each of containment units 18 and 18a, one or more mirrored surfaces are used to provide a view of fluid delivery set 15 without creating an unshielded direct line between the viewer and the fluid delivery set 15 (or other radioactive source). There is no need to provide a transparent shield (for example, lead shielded glass) over view ports 162 or 172 because the lack of an unshielded direct line of sight between the viewer and fluid delivery set 15 prevents exposure to radiation. Elimination of leaded glass can be advantageous as such glass is often expensive and heavy and can sometimes diminish or degrade a view.

In the case of injection of a radiopharmaceutical, positioning a pressurizing unit or chamber such as syringe 60 within dose calibrating unit 200 such as the Capintec CRC-15PET dose calibrator available from Capintec, Inc. of Ramsey, N.J., which measures the total radiation of the volume of radiopharmaceutical enclosed within the pressurizing chamber, shields administering personnel from radiation and enables delivery of a known volume of the radiopharmaceutical having a known radiation level (as measured directly by dose calibrating unit 200). The accurate control of injection volume and flow rate provided by powered injector 70 enables automatic injection of a calculated volume of fluid (using for example processing unit 71 of injector 70) that will provide the level of radiation necessary, for example, for a PET or SPECT image given the measured radiation of the total volume of radiopharmaceutical contained within syringe 60 provided by dose calibration unit 200. Thus, it is no longer necessary to calculate and wait for the precise moment in time when radioactive decay has brought the level of radiation of a volume of radiopharmaceutical to the desired level, thereby saving time and reducing the complexity of the injection procedure.

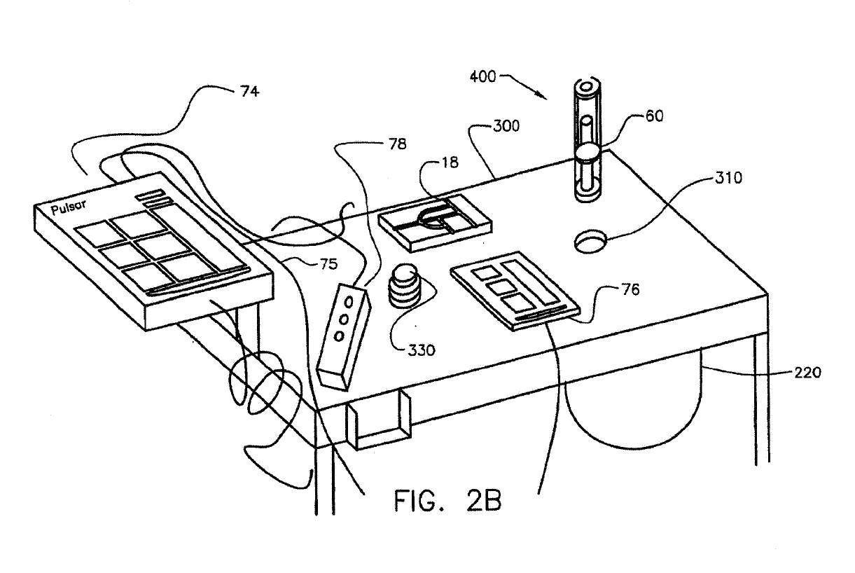

FIGS. 2-4C illustrate one embodiment of a setup for system 10 as described above. In this embodiment, a PULSAR injector available from Medrad, Inc. of Indianola, Pa. was used. Injection head 72 was separated from control unit 74 as described in U.S. Provisional Patent Application Ser. No. 60/167,309, filed Nov. 24, 1999, U.S. patent application Ser. No. 09/721,427, filed Nov. 22, 2000 and U.S. patent application Ser. No. 09/826,430, filed Apr. 3, 2001, all assigned to the assignee of the present invention. Injection head 72 is slidably positioned in general alignment with an opening 204 in dose calibration unit 200 on a generally vertical slide bar or stand 220 via a clamping extension 224. Injector 70 also includes a first remote control unit 76 for communicating data/instructions such as injection volume and flow rate into control unit 74 remotely (via, for example, communication line 75). Further, injector 70 includes a second remote control unit 78 for remote manual control of drive member 79 of injector 70. The function of first remote control unit 76 and second control unit 78 can be combined. On currently available PULSAR injectors, manual controls for drive member 79 are positioned upon injector head 72. However, to prevent undesirable exposure to radiation in system 10 of the present invention, such controls are preferably also positioned remotely from injector head 72. Saline source/syringe 20 can also be controlled via injector 70 through a second injector head (not shown) as described, for example, in U.S. Provisional Patent Application Ser. No. 60/167,309, filed Nov. 24, 1999, U.S. patent application Ser. No. 09/721,427, filed Nov. 22, 2000 and U.S. patent application Ser. No. 09/826,430, filed Apr. 3, 2001.

In the embodiment of FIGS. 2A through 4C, system 10 is positioned upon a cabinet stand 300. Slide bar 220 extends generally vertically from cabinet stand 300. Cabinet stand 300 includes a passage 310 formed therein through which syringe 60 can pass to enter dose calibration unit 200. Cabinet stand 300 also preferably includes a second passage 320 through which pharmaceutical source 40 can pass to be deposited within container 44. A cap 330 can be provided to seal container 44. In the embodiment of FIGS. 2A through 4C, first passage 310 is preferably oriented such that radiation emanating therefrom is directed generally vertically toward the ceiling (or in another suitable direction) to reduce the likelihood that personnel within the room of the injection procedure will be exposed to such radiation.

Injector head 72 is oriented in a generally vertical, downward direction on slide bar 220 to position syringe 60 within dose calibrating unit 200. To ensure that air is purged from a syringe, however, injector heads are typically positioned such that the exit, or tip, of the syringe is oriented upward during purging. As air is less dense than other injection media and saline flushes, the air rises to the syringe tip or exit and is readily purged by, for example, forcing a small amount of fluid from the syringe. To enable a generally vertical orientation of syringe 60 with syringe tip 64 oriented upward in the present invention, a syringe adapter 400 was used.

Syringe adapter 400 attaches to injector 70 in preferably the same manner as syringes are attached thereto. Attachment adapters can be used as known in the art to facilitate such attachment. Adapter 400 can, for example, be removably attached to injector 70 via flanges 412 on an attachment member 410 that cooperate with retaining slots in injector 70 (not shown) as described in U.S. Pat. No. 5,383,858, assigned to the assignee of the present invention, the disclosure of which is incorporated herein by reference.

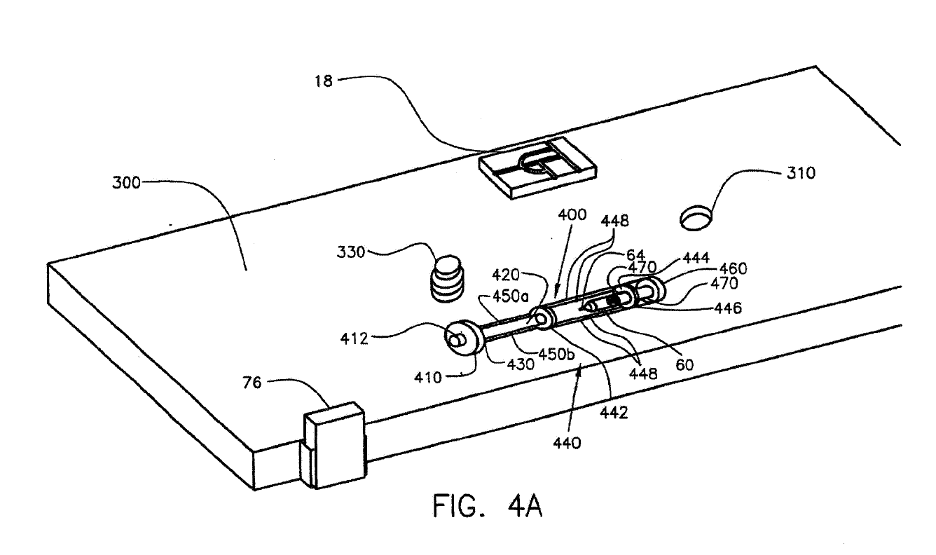

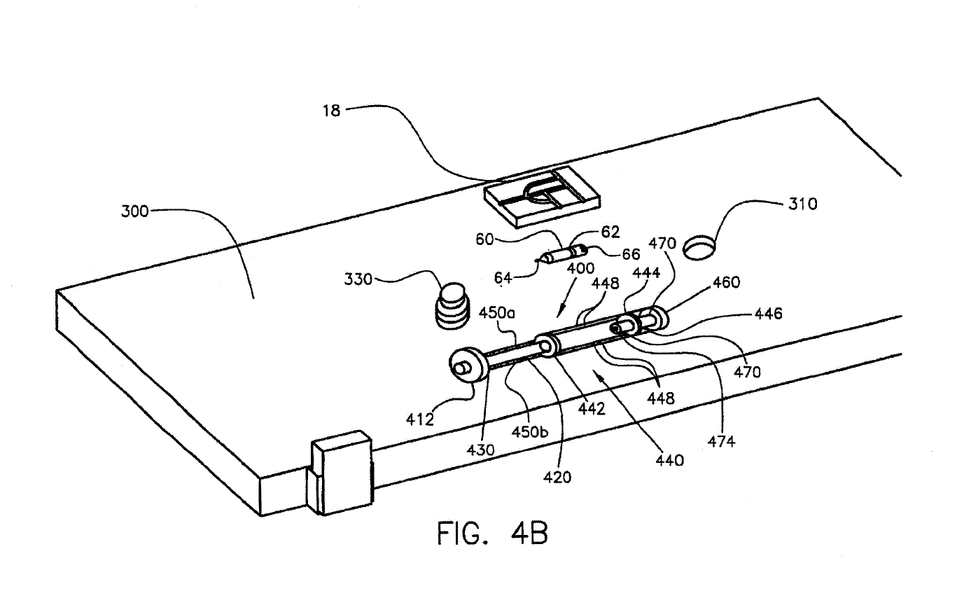

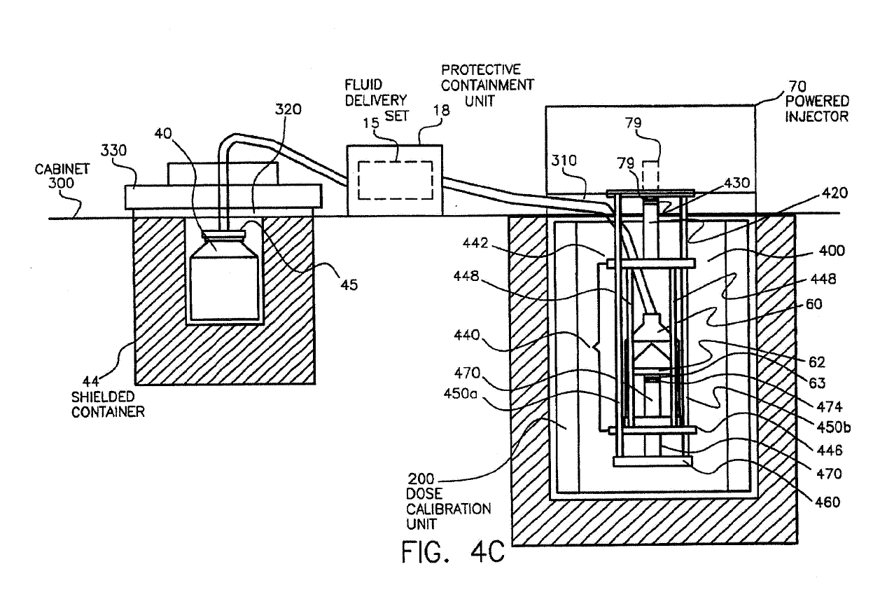

Adapter 400 includes a drive extension 420 that removably connects to drive member 79 of injector 70 via an attachment member 430 that can, for example, include capture members that cooperate with a drive member flange 79′. Drive extension 420 attaches to a syringe carriage 440 at an upper plate member 442 of syringe carriage 440. Syringe carriage 440 is slidably disposed upon adapter 400 via slide bars 450a and 450b that extend from the rear surface of attachment member 410 to a fixed, lower base 460. Syringe carriage 440 includes a syringe attachment member 444 attached to a lower plate member 446 of syringe carriage 440. Upper plate member 442 and lower plate member 446 are connected via connecting members 448 (for example, metal or plastic bars). Syringe attachment member 444 can include slots (not shown) that cooperate with flanges 66 on a rear portion of syringe 60 to removably attach syringe 60 to syringe carriage 440 as illustrated in FIGS. 4A and 4C (as described, for example, in U.S. Pat. No. 5,383,858). Via syringe carriage 440, the barrel of syringe 60 is slidable in an upward and downward direction on adapter 400.

Adapter 400 further includes a plunger extension 470 that includes a plunger attachment including, for example, a flange 474 that cooperates with capture members 63 on the rear of plunger 62 to removably connect plunger extension 470 to plunger 62. Adapters as known in the art can facilitate connection of plunger extension 470 to various plungers. Plunger extension 470 maintains plunger 62 in a fixed position relative to base 460 and injector head 72. By upward and downward movement of syringe carriage 440 (via injector drive member 79 and drive extension 420), the position of plunger 62 within syringe 60 is changed. For example, advancing drive member 79 causes the barrel of syringe 60 to move downward and causes a corresponding or relative advancement of plunger 62 toward syringe tip 64, thereby causing fluid to be expelled from syringe 60. Upward movement (or retraction) of drive member 79 causes the barrel of syringe 60 to move upward and corresponds to retraction of plunger 62 relative to syringe tip 64, thereby drawing fluid into syringe 60.

An extending syringe adapter, such as adapter 400, enables use of commercially available injector systems and commercially available dose calibrators in the system of the present invention without substantial modification. The use of adapter 400 is transparent to the injector control software/hardware as no change and/or recalibration of the controlled movement of drive member 79 of injector 70 is required.

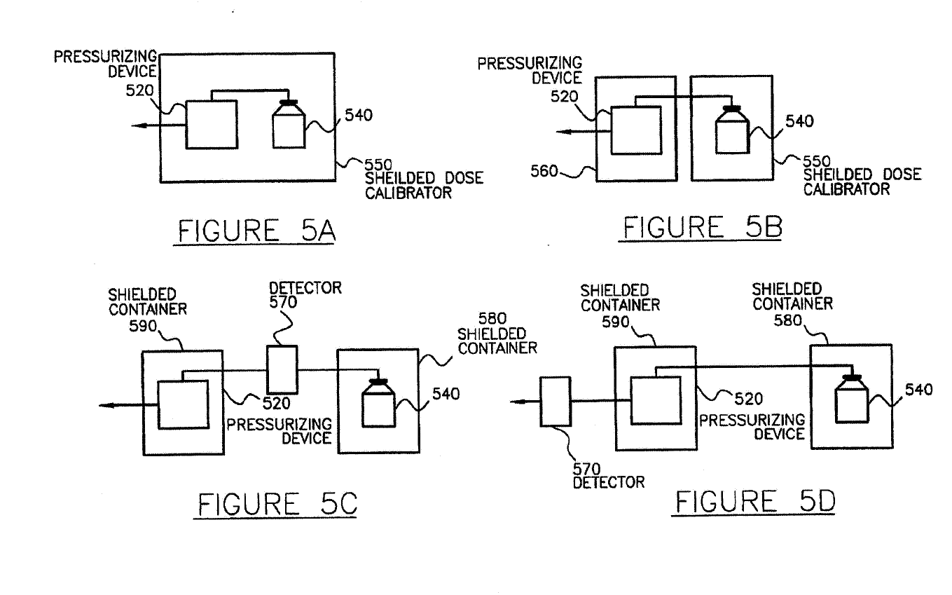

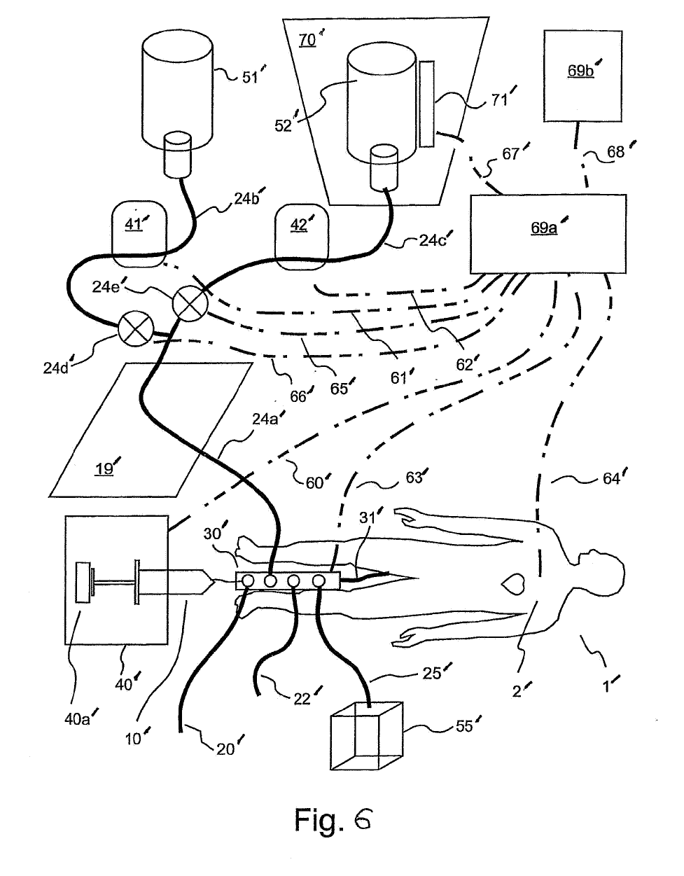

FIGS. 5A through 5D illustrate several other embodiments of the present invention for providing dose calibration generally in real time. In FIG. 5A, a pressurizing device 520 (for example, a syringe in communication with a powered injector) and a radiopharmaceutical source 540 are positioned within a dose calibrator 550. In FIG. 5B, radiopharmaceutical source 540 is placed in a dose calibrator 550′, while pressurizing device 520 is placed in a shielded enclosure 560. In the embodiment of FIGS. 5C and 5D, radiation level detectors are placed in operative connection with flow lines (for example, tubing). In FIG. 5C, a radiation detector 570 is placed in line between radiopharmaceutical source 540 (enclosed within a shielded container 580) and pressurizing device 520 (enclosed within a shielded container 590). In FIG. 5D, a radiation detector 570′ is placed in line with the exit of pressurizing device 520. In general, the flow rate through the line in operative connection with radiation detector 570 or 570′ is known. The radiation level of a particular dose is thus easily measured using radiation detectors 570 and/or 570′.

FIG. 6 illustrates a conventional system for dispensing hazardous pharmaceuticals, as disclosed in International Patent Application No. WO 2004/091688 (Medrad, Inc.; Uber et al.) This publication (“WO '688”), and any U.S. or non-U.S. patents, patent applications or patent applications in its family, are fully incorporated by reference as if set forth in their entirety herein. By way of a set of completely illustrative and non-restrictive examples, the systems broadly contemplated and disclosed in WO '688 and its family constitute suitable environments in which embodiments of the present invention may be employed. One distinction of FIG. 6 as compared to FIGS. 1A-5B is in the use of a pump arrangement, rather than injector arrangement, to propagate radiopharmaceutical.

As with the '319 patent, though the embodiments of the present invention may be employed in a wide variety of settings and environments, WO '688 and its family may be referred to for useful background information for better appreciating the embodiments of the present invention and their manner of functioning. FIG. 6, as discussed below and included with the instant application, is derived from FIG. 2 of WO '688.

FIG. 6 illustrates a conventional fluid delivery system. In the system of FIG. 6, patient 1′ has a catheter 31′ inserted via a femoral approach into the patient's heart 2′. Catheter 31 is connected to a manifold 30′ to enable injection of various fluids. Syringe 10′ can be filled with contrast from line 20′ and then injected. There, syringe 10′ is operated by a mechanical injector 40′ including a piston 40a′ that pushes or pulls on the syringe plunger extension, thereby moving fluid in and out of syringe 10′. An injector or syringe pump, such as the ProVis angiographic injector available from Medrad, Inc. of Indianola, Pa., can, for example, be used as pump 40′.

Pump 42′ delivers a radiopharmaceutical or other drug. In this embodiment, the drug remains in its container 52′ and is pumped from the container by a peristaltic pump 42′. The drug flows though tubing 24c′ and then tubing 24a′ into the manifold and thence into patient 1. Tubing 24c′ and 24a′ can, for example, be microbore tubing to minimize the amount of fluid or dead space in the tubing itself. In the embodiment of FIG. 6, pump 42′ and the drug containing apparatus are outside the sterile field. The fluid is brought into the sterile field through sterile tubing 24a′. Drug container 52′ can be any container which preserves the sterility and utility of the drug including, for example glass bottles, bags, carpules, or prefilled syringes. If container 52′ (or any other fluid container in the system) is rigid, a vacuum will be created as fluid is pulled out. There are several methods to eliminate this problem. For example, air can be injected into the container before removal of the fluid, or the needle or spike used to remove the fluid can be vented with a hydrophobic filter or with a one way valve and filter that allows sterile air to enter the container as the fluid is removed but prevents any leakage of the fluid.

A biohazard containment or enclosure 70′ enables spiking and withdrawal of the administered drug (e.g., radiopharmaceutical) from drug container 52′ outside of the pharmacy and, indeed, outside of a hood. One end of fluid path element 24c′ penetrates and is sealed to biohazard enclosure 70′. The spike, needle, or other mechanism for making fluid connection to drug container 52′ is inside biohazard enclosure 70′ and is sheathed to protect the operator and enclosure 72′. During use, biohazard enclosure 70′ is opened, and drug container 52′ is placed inside. Then, biohazard enclosure 70 is sealed and container 52′ is connected to fluid path 24c′ using gloves or other flexible handling devices that operate through the walls of biohazard enclosure 70′. If biohazard enclosure 70′ is flexible, it does not need to be vented. If it is rigid or semi-rigid, it preferably incorporates a vent, which is preferably adapted or designed to contain any aerosolized biohazardous material. The vent can incorporate activated charcoal or a zeolite material if it is necessary or desired to contain drug vapors as well. The in-suite biohazard enclosure 70′ of the present invention saves considerable time, labor and expense by eliminating the syringe filling steps in the pharmacy. Biohazard enclosure 70′ can for example, include a Captair Field Pyramid glove box available from CAPTAIR LABX, INC. of North Andover, Mass.

As shown in FIG. 6, a thermal device 71′ can be in thermal connection with the container 52′. Thermal device 71′ can, for example, be a thermoelectric heater/cooler that can maintain the drug in a frozen state and then controllably heat the drug to either room temperature, body temperature, or another temperature at a controlled rate. Thermal device 71′ is connected to control unit 69a′, which coordinates its operation. Thermal device 71′ can, for example, help maintain the drug at a reduced temperature through passive insulation or through active chilling (for example, with dry ice or with a mechanical refrigerator). Heat can be provided in many ways including, but not limited to, a resistive heater, microwaves, chemical reaction(s), material phase change(s), or hot air.

Pump 42′ can provide steady consistent flow over extended periods of time (for example, over minutes) much better than a human pushing a syringe plunger. The consistent flow provided by pump 42′ reduces the risk associated with operator fatigue and/or mistakes. Also, by making the connection in a protected way, and then throwing away, as a unit, fluid path 24′, containers 51′ and 52′, enclosure 70′, and other fluid path elements, there is no opening of the fluid path that could allow the biohazardous material to escape into the environment.

Saline, other flushing fluid or another non-hazardous drug can be stored in container 51′. Flow is driven or caused by pump 41′. The flushing fluid flows through tubing 24b′ and 24a′, into manifold 30′ and thence into patient 1′. In certain gene therapy procedures, the initial flush flow rate is preferably the same as the drug flow rate and preferably begins immediately after the flow of the drug is stopped, because it is used to flush drug out of the fluid path into patient 1′. The saline can also be pumped simultaneously with the drug to provide dilution of the drug if that is advantageous. Rapid alternations between saline and drug delivery can also produce a dilution effect with the fluids mixing as they traverse the remainder of the fluid path. Additionally, in situations where two or more of the possible multiple fluids are incompatible, the flushing fluid can be used to separate the incompatible fluids before delivery to the patient. For example, some X-ray contrasts are incompatible with some gene therapy drugs.

Pumps 41′ and 42′ can be one of many commercially available pumps. For example, a suitable pump is the “CONTINUUM” pump available from Medrad, Inc. of Indianola, Pa. The “PEGASUS” series of pumps available from Instech Laboratories, Inc. of Plymouth Meeting, Pa., can also be used in some applications. Depending upon the details of the procedure and the number of fluids to be used, multiple hazardous fluid pumps with containment chambers and or multiple non-hazardous fluid pumps can be used.

Where fluid lines 24b′ and 24c′ come together to start segment 24a′, it can be useful to have one or more spring-loaded one way valves or electrically controlled valves 24d′ and 24e′, so that there is no flow or diffusion of one fluid into another fluid. A similar use of valves is, for example, found on the disposable fluid path used with the “SPECTRIS” MR injectors available from Medrad, Inc. to prevent diffusion mixing of MR contrast into the flushing fluid.

Another feature that can increase ease of use and safety is waste container 55′ illustrated in FIG. 6, which is connected to manifold 30′ via tubing 25′. Waste container 55′ can, for example, be a sealed, initially collapsed bag, or a rigid or semi-rigid container with a filtered vent. When fluid lines are first connected, they can be dry (full of air.) Because it is generally bad to inject air into a patient's blood vessels, it is necessary to prime or purge the fluid lines, that is, to push fluid through the lines to remove the air. To eliminate the chance that any biohazardous material is released into the environment, first contrast syringe 10′ and manifold 30′ can be primed, either into waste container 55′ or using the procedures currently done. Then the biohazardous drug is primed through 24c′ and just a little bit beyond into tube 24a′. Then the flush fluid is primed through 24b′ and 24a′ all the way into waste container 55′. In this manner, no biohazardous material is released during the purging process. In an alternative embodiment, the fluid path can be primed with, for example, saline prior to connecting the fluid path to container 52′. Such “prepriming” is discussed in U.S. Patent Application Publication No. 2003-0004463, filed Jul. 4, 2002, assigned to the assignee of the present invention, the disclosure of which is incorporated herein by reference.

Dashed lines 60′, 61′, 62′, 63′, 64′, 65′, 66′, 67′, and 68′ in FIG. 6 represent communication paths for information or control transmission or transfer. In the system of FIG. 6, control unit 69a′ communicates with the system pumps and the patient. Control unit 69a′ also preferably includes a user interface 69′b through which the operator can monitor, program, or control all the associated devices. User interface 69b′ allows the operator to input settings or controls, and to assess the condition and operation of the system. In one embodiment, user interface 69b′ includes a display with a touch screen as known in the computer arts. Portions of user interface 69b′ can optionally include a foot pedal, hand switch, voice recognition, voice output, keyboard, mouse, and/or an LCD display. Control unit 69a′ can, for example, include a personal computer with a keyboard, speakers, and display that serves as user interface 69b′. Software such as “LABVIEW” available from National Instruments of Austin, Tex., is, for example, capable of collecting data and creating sophisticated control strategies based upon that data and may be incorporated into control unit 69a′.

Lines 60′, 61′, and 62′ communicate with system pumps 40′, 41′, and 42′, respectively. Line 63′ communicates with manifold 30′ so that the proper fluid path is open at the proper time. Lines 65′ and 66′ can operate valves 24d′ and 24e′ respectively, if they are controlled valves rather than spring loaded valves. Line 64′ is shown schematically to bring heartbeat information from patient 1′ to control unit 69a′. An instrument (not shown) can be provided that acquires the signal and conditions or operates on it before outputting it to control unit 69a′. The instrument can, for example, be an ECG monitor, a blood pressure monitor, a pulse oximeter, image segment or region of interest extractor, or other device. If control unit 69a′ incorporates, for example, a data acquisition card (available, for example, from National Instruments) with sufficient isolation, no additional instrument is necessary. In situations where the target is an organ other than the heart, the instrument can monitor some physiological parameter or imaging aspect related to that target organ. An example is monitoring respiration where the parameters of interest are respiration rate, tidal volume and end tidal volume. Other examples are peristaltic contraction of the intestines or voluntary or stimulated contraction of muscles.

Communications and control in the systems contemplated herein can have various levels of sophistication based upon design, verification, economic, and usability considerations. A simple level involves centralized start/stop timing or synchronization between two or more devices. A next level can, for example, be centralized programming of one or more pumps to improve operator or user convenience. A next level can, for example, involve a common programming interface for all pumps. A next level can, for example, include standard protocols involving various synchronization strategies and allowing the operator to save and recall customized protocols. The systems of the present invention provide great flexibility for designers to meet user needs.

It certain situations, it can be advantageous to have contrast injector or pump 40′, similar to that described in U.S. patent application Ser. No. 09/982,518, filed on Oct. 18, 2001, assigned to the assignee of the present invention, the disclosure of which is incorporated herein by reference, be the primary controller, performing many of the functions of control unit 69a′. In this embodiment, pumps 41′ and 42′ communicate to contrast pump 40′ and all the operations described herein are achievable. The additional fluid delivery systems could be considered as accessories for the contrast pump 40′.

To check for proper fluid line purging, air detectors such as those available from Introtech of Edgewood, N.Y., can be included at various places along the fluid path.

While the embodiments of the present invention described above include pumps that can be applied for the delivery of all the fluids related to a procedure, for either cost or historic preference, perception, or feelings of wanting to be in control, some of the pumping functions can be performed manually while others are performed mechanically. Specifically, many doctors prefer the manual “feel and control” of conducting the contrast injection. In this case only pumps 41′ and 42′ are used. Alternatively, mechanical delivery can be used and tactile feedback provided to the doctor to simulate the “feel and control” of manual operation. Tactile feedback is discussed in U.S. Pat. No. 5,840,026 and in U.S. patent application Ser. Nos. 09/982,518 and 10/237,139, assigned to the assignee of the present invention, the disclosure of which are incorporated herein by reference.

Finally, by way of additional background references (again, for illustrative and non-restrictive purposes), International Patent Application Nos. WO 2006/007750 (Universität Zütrich; Buck et al.) and WO 2004/004787 (UniversitéLibre de Bruxelles—Hôpital Erasme; van Naemen et al.) illustrate other conventional systems for dispensing hazardous pharmaceuticals, and are particularly directed to the dosing of such pharmaceuticals. These publications (Buck and van Naemen, respectively) are fully incorporated by reference as if set forth in their entirety herein. By way of a set of completely illustrative and non-restrictive examples, the systems broadly contemplated and disclosed in Buck and von Naemen constitute suitable environments in which embodiments of the present invention may be employed.

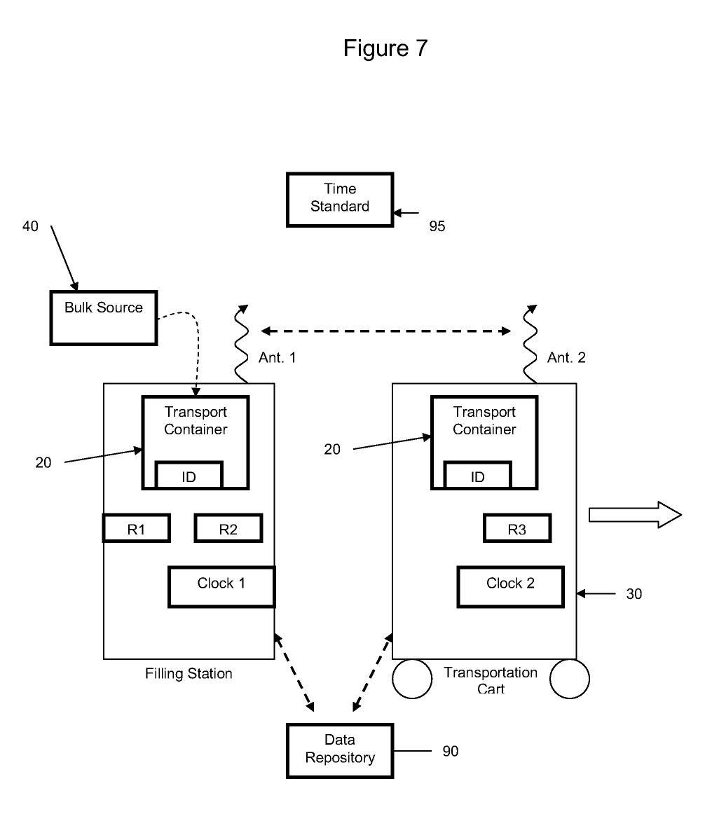

The disclosure now turns to a discussion of embodiments of the present invention as illustrated in FIG. 7 and as may be employed in any environment embraced within the scope of the discussion hereinabove or in any of a very wide variety of analogous environments. Again, the environments broadly embraced within the scope of FIGS. 1A-6, and Buck and van Naemen, as discussed hereinabove, are provided by way of illustrative and non-restrictive examples.

Broadly contemplated herein, in accordance with at least one presently preferred embodiment of the present invention, are systems, devices, and methods for more accurately determining a radiopharmaceutical dose administered to a patient by relying on a time factor. Particularly, broadly contemplated herein is the administration of a dose on the basis of an elapsed time from when a dose was last accurately measured in the past (e.g., when initially dispensed) to when it is injected into the patient.

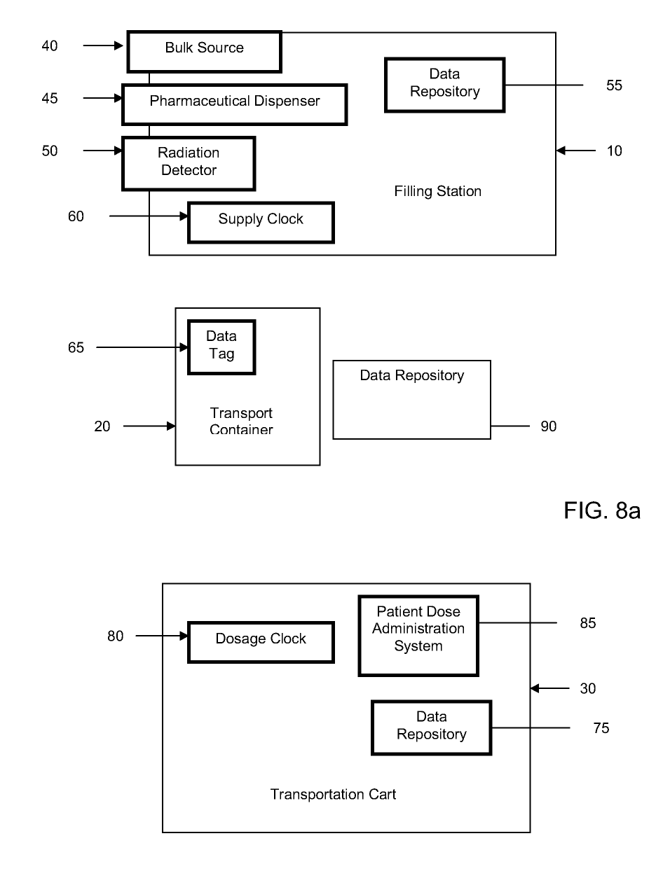

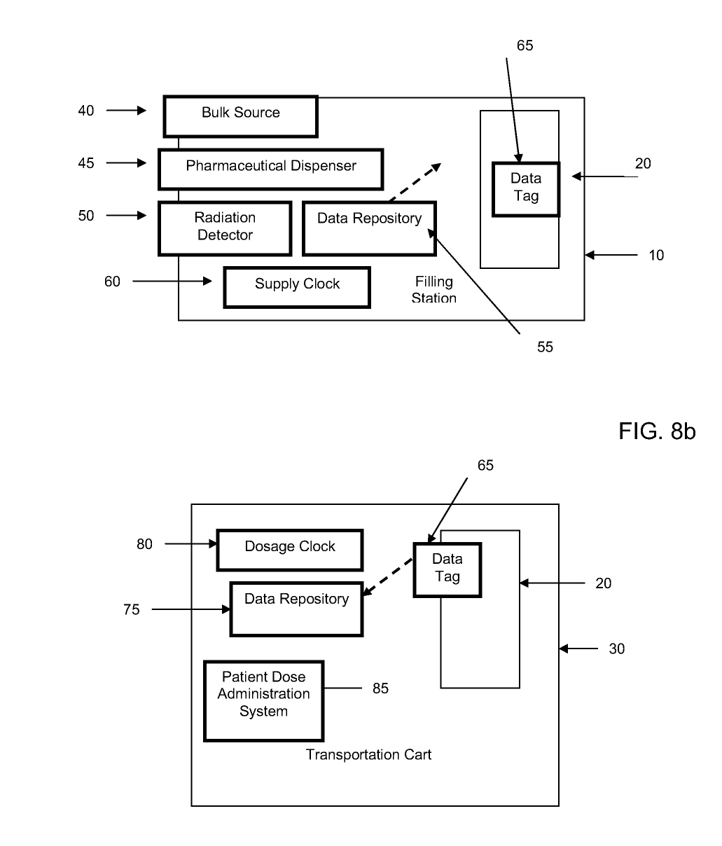

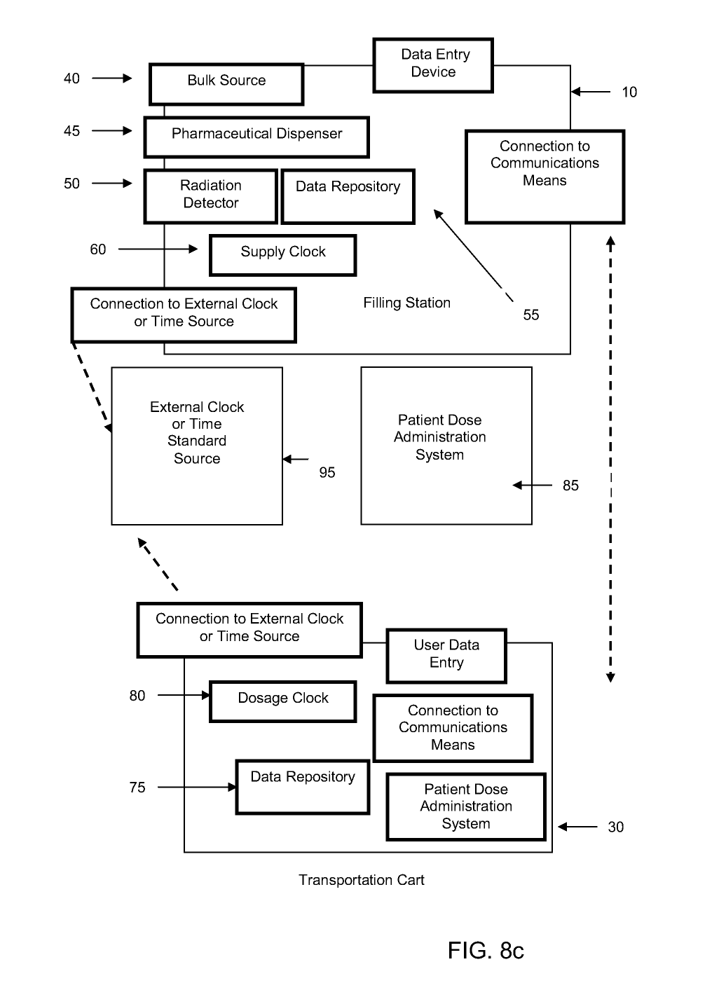

FIGS. 7 and 8a-8c illustrate several embodiments of a dosing system of the present invention. The dosing system comprises radiopharmaceutical filling station 10, transport container 20 to hold a portion of the radiopharmaceutical drug, and transportation cart 30.

Filling station 10 includes bulk source 40 of a radiopharmaceutical connected to pharmaceutical dispenser 45, radiation detector 50, data repository 55, supply clock 60 and removable transportation container 20. Bulk source 40 contains a radiopharmaceutical (drug) that has a half-life indicative of the rate of radioactive decay. Pharmaceutical dispenser 45 dispenses some portion of the radiopharmaceutical from bulk source 40 into transport container 20.

Radiation detector 50 reads or measures the radioactivity of the drug portion dispensed into transport container 20, as a “radioactivity measure.” The radioactivity measure is accurate at the time of dispensing. The radioactive detector 50 may be any appropriate measuring device well-known in the art.

Further, supply clock 60 provides the time at which the drug portion is dispensed into transportation container 20 or “fill time.” Alternatively, filling station 10 may be in communication with a separate clock or time standard, not directly associated with filling station 10. FIG. 7c illustrates this alternate embodiment, where the time of dispensing the drug portion is obtained from this separate clock or time standard source 95. Non-limiting examples include, a wireless time standard such as those based at the U.S. Time Service of the U.S. Naval Observatory or the National Institute of Science and Technology (NIST). If the system is to be utilized outside of the U.S., similar time standards (such as governmental time standards) in other countries can well be employed.

Data repository 55 reads or receives data related to the drug or delivery, including but not limited to the fill time or radioactivity measure. It should be appreciated that data repository 55 may also be located remotely (as data repository 90), either additionally or alternatively.

Transport container 20 is a removable receptacle that is in communication with filling station 10 to receive at least a portion of the dispensed radiopharmaceutical. Transportation container 20 includes data tag 65 to which data may be recorded and/or written. Data tag 65 can include any appropriate date means, including but not limited to, an RFID chip, a barcode, or other means of recording/writing data. When transportation container 20 is at filling station 10, information data may also be recorded on data tag 65. The information data may include any information related to the drug and/or delivery, including but not limited to, drug name, drug half-life, date of filling, drug characteristics, type of radionuclide, measured radioactivity of the dispensed drug portion, time that the transportation container is filled with a portion of the drug, or any other type of information related to characteristics, delivery or dispensing of the drug.

The half-life of the pharmaceutical can also be obtained by a look-up table so that the radioactive decay may be calculated between the filling time of dispensing the dose into the transportation container and the injection time when the dose is injected into the patient. Once the transport container has been filled with the drug portion at the filling station, and the data recorded on at least the data tag or other data storage device, it is ready for delivery to the patient.

In addition to storing, exchanging or reading data via data tag 65 of transportation container 20, filling station 10 may also incorporate means for the operator to include drug and delivery related information into data tag 65. For example, the operator can use any appropriate input device, including but not limited to, a keyboard. With such a device, the operator can enter information about the radiopharmaceutical into the dosing system at any suitable time.

The dosing system also includes transportation cart 30 that may be used with transportation container 20. Transportation cart 30 includes data repository 75, patient dose administration system 85 and dosage clock 80. It should be appreciated that any of the various components of the system can be in communication with each other.

In the preferred embodiment, transportation cart 30 is a mobile device that can be transported to a patient at some distance from filling station 10. Further, transport container 20, once removed from filling station 10, may be removably docked into transportation cart 30.

In the preferred embodiment, dosage clock 80 is synchronized to supply clock 60 associated with the filling station. Such synchronization may be accomplished when transportation cart 30 is in proximity to the filling station. Alternatively, such synchronization may be accomplished remotely, through a wireless communication or internet connection with the filling station or via a separate clock (or time standard). Further, the synchronization may occur once, continuously or intermittently, as needed to provide the most accurate timekeeping.

Data repository 75 obtains information from a variety of sources, including at least data tag 65 and/or data repositories 55,90.

Information may also be read and/or processed by the patient dose administration system 85. For example, information is preferably read immediately before the drug is administered to the patient. Patient dose administration system 85 can calculate the radioactivity in transportation container 20 depending on a variety of variables, including at least the fill time associated with the filling of the transportation container, the current time (synchronized to the time that the transportation container was filled), the measured radioactivity of the drug at the fill time, and the known half-life of the drug. Further, patient does administration system 85 records and/or writes information associated with the drug administered to the patient, or other relevant data to any appropriate data source, including but not limited to, data repositories 55, 90, 75. The communication from data repositories 75 may be enabled by any suitable means, including but not limited to, wireless RF, or via an internet connection.

Further, administration station 30 may obtain or exchange information via alternate means. For example, administration station 30 may be in direct communication with filling station 10 to exchange information. Administration station 30 also may be similar to filling station 10 by including an input means, such as but not limited to a keyboard, for the user to incorporate additional information into the data repository or data tag.

The dosing system of the present invention may also include data repository 90 provided for recording or sending all pertinent data discussed hereinabove. Data repository 90 could be an additional component remote from filling station 10 or administration system 30, or could be integral to either filling station 10 or administrative system 30. The recorded information may include, but is not limited to, time the transport container is filled, level of radioactivity contained in the container immediately after the container is filled, name of the radionuclide of the radiopharmaceutical or its decay half-life, and identification information of the transportation container recorded from the data tag.

To the extent that a data repository is remote with respect to either the filling station or administration station or both, a suitable communication link (such as the wireless link discussed hereinabove, or an alternative arrangement such as an optical/infrared communications link) could be employed to exchange data with the data repository. All such data exchanged can be encrypted to secure the data against unauthorized reading, and the repository itself may be secured against deliberate or unintended alteration.

The preferred method for using the system is described as follows. Transport container 20 is initially placed in filling station 10 where it receives a portion of the radiopharmaceutical drug or dispensed dose. The dose dispensed into transportation container 20 may either be derived solely from the bulk supply, or may be mixed with a non-radioactive diluent.

Radiation detector 50 associated with the filling station measures the radioactivity of the dispensed dose. If mixed with diluent, the radioactivity of the dose in the transportation container 20 can be measured after it has been mixed and filled.

The information related to the radioactivity is recorded in at least data tag 65. Additionally, this information may be communicated to other data storage, including data means 55, data means 75 or data repository 90. Once the dose has been produced, the time source, whether the supply clock or time standard, provides a fill time. The fill time can be then recorded to at least the data tag 65, data repository 90 or any other appropriate storage unit.

Transport container 20 is thereafter placed in transportation cart 30. The dosage clock 80 associated with transportation cart 30 is synchronized with supply clock 50 of filling station 10 or any appropriate time source, when container 20 is placed in administration station 30. Alternatively, dose clock 80 can be synchronized anytime after or before the does to provide to transportation container 20.

The administration station 30, containing transport container 20 is moved to the patient, and the drug may be delivered to the patient. Patient dose administration system 85 reads the data from at least data tag 65, preferably immediately before the radiopharmaceutical is delivered, as well as the current time from dosage clock 80. However, it should be appreciated that data may also be obtained from remote sources such as data device 55, data means 75, data repository 90 or external clock 95. Once sufficient data is obtained, patient dose administration system 85 calculates the dose to administer to the patient based on the radioactivity measure of the dispensed dose initially portioned into transport container 20, the current time from the synchronized time source, the fill time, and the half-life of the drug.

FIG. 7 illustrate an alternate embodiment where various reading devices and clocks are utilized in the filling station and transportation cart. The base unit or filling station is configured to hold the pharmaceutical storage unit or transport container (“T.C.”) for being filled from a bulk supply. The pharmaceutical storage unit is then placed in a shielded patient dosing unit or transport station (or cart). Preferably, the transport container has an identification arrangement (“ID”) such as a barcode or resonant frequency tag that can easily be read by suitable apparatus. Accordingly, the filling station preferably has such a reader r1 for this purpose.

Preferably, the filling station also has another data collection arrangement r2 for reading, ascertaining or measuring the radiation dose (radioactivity level) present in the transport container. Examples of such data collection arrangements abound and are well-known to those of ordinary skill in the art. Furthermore, the filling station also preferably includes a clock (“clock 1”) which establishes a timepoint at which the transport container is filled with radiopharmaceutical.

Accordingly, in accordance with a preferred embodiment of the present invention, when the transport container is filled at the filling station, the radiation dose is recorded along with the timepoint of filling. The transport station or cart, for its part, will also preferably include a clock (“clock 2”) which is synchronized with clock 1. Upon being loaded into the transport station or cart, another reader (r3) may read the ID information of the transport container (though this can also be read by a reader present at the patient dose administration system [not shown in FIG. 7]). The ID information is preferably read immediately before radiopharmaceutical is administered to a patient.

Preferably, a suitable communication link is provided between the filling station and transport station or cart, for instance, via wireless communication between antennae at the filling station and transport station or cart (“ant. 1” and “ant. 2”, respectively). This communication link permits data, as discussed below, to be exchanged in a manner to readily ascertain the radiation dose administered to a patient. As such, embodiments of the present invention serve to obviate the need to directly measure a dose of radiopharmaceutical administered to a patient when it is being administered to the patient. By alleviating the need for such direct measurement a second time (in addition to the measurement normally taken at the filling station), a much less expensive system is attainable than the norm.

Once at a patient dose administration system, the dose in the transport container is preferably calculated based on the time associated with the container and the known half-life of its contents. Thus, a calculation is preferably made which takes into account two distinct timepoints (i.e., the time of filling and the time at which it is desired to ascertain the radiation dose again, such as when it is to be administered to a patient) and the known half-life of the radiopharmaceutical, so as to clearly establish the degree to which the radiopharmaceutical may have decayed and thus lost potency.

Preferably, the filling station and transport cart clocks 1 and 2 can reference a wireless time standard such as those based at the U.S. Time Service of the U.S. Naval Observatory or the National Institute of Science and Technology (NIST). If the system is to be employed outside of the U.S., similar time standards (such as governmental time standards) in other countries can well be employed.

As is well-known, at times it may be desirable to add a non-radioactive diluent to the transport container such that the total volume in the container comprises the volume of the radiopharmaceutical plus that of the added diluent. If this is the case, the radioactivity of the contents of the transport container can be measured after it has been filled.

Preferably, the ID information on the transport container will provide information on the type of radionuclide that comprises the radiopharmaceutical. Ultimately, the half-life of the radiopharmaceutical can be obtained by a look-up table in order to help calculate the radioactive decay between the two timepoints mentioned above. Alternatively, such information on the radiopharmaceutical can be entered by an operator at any suitable time.

The system of the present invention may also include data repository 90 provided for recording all pertinent data discussed hereinabove. Data repository 90 could be an additional component remote from filling station 10 or administration system 30, or could be integral to either filling station 10 or administrative system 30. The recorded information may include, but is not limited to, the time the transport container is filled, the level of radioactivity contained in the container immediately after the container is filled, the name of the radionuclide of the radiopharmaceutical or its decay half-life, and the identification information of that portable container recorded from the ID arrangement of the container. To the extent that a data repository is remote with respect to either the filling station or administration station or both, a suitable communication link (such as the wireless link discussed hereinabove, or an alternative arrangement such as an optical/infrared communications link) could be employed to exchange data with the data repository. All such data exchanged can be encrypted to secure the data against unauthorized reading, and the repository itself can be secured against deliberate or unintended alteration.

By way of improvements enjoyed herein, in contrast with other radiopharmaceutical delivery devices (e.g. for FDG) tend to rely on an expensive and bulky radiation dose calibrator to measure the dose directly before administering it. In contrast, clocks as employed herein tend to be cheaper and contribute to a more accurate measurement (especially if the clocks are tied to a known standard).

By way of a general overview of possible operating environments of the present invention and their components, it is to be appreciated that, as used herein in connection with several of the various embodiments of the present invention, the term “pump” includes all means of causing a controlled fluid flow, including controlled pumps or pressure sources and regulators, for example peristaltic pumps, gear pumps, syringe pumps, electrokinetic pumps, gravity, compressed gas, controlled gas evolving devices, spring pumps, centripetal pumps or any system which does not require continuing human exertion of motive force when the fluid is flowing. A number of the aspects of the present invention can also be advantageously applied to hand activated pumps as well.

It is to be appreciated that the systems, devices and methods of the present invention can be used in a very wide variety of drug delivery and therapeutic procedures. In general, the systems, devices and methods of the present invention are particularly suited for use in connection with any hazardous pharmaceutical or substance to be injected into a patient (human or animal). Exemplary methods of administering hazardous pharmaceuticals include intra-arterial, intravenously, intramuscularly, subcutaneously, by respiration into the lungs, and transdermally. Even pharmaceuticals that are not considered to be extremely hazardous can be beneficially administered via systems broadly contemplated herein and provide hospital personnel additional protection against adverse effects.

To the extent that systems of the present invention can be applicable to radiotherapy drugs or pharmaceuticals wherein the drug or pharmaceutical itself is radioactive, it is to be appreciated that, as clear to one skilled in the art, maintaining complete containment of radiotherapy pharmaceuticals promotes safety. If the drug or pharmaceutical is radioactive, the use of radiation absorbing or leaded Plexiglas shielding will help protect the operator and patient from unnecessary radiation dose. Designers skilled in the art of radiation shielding can readily specify the thicknesses needed. Containment of radiotherapy pharmaceutical is discussed in U.S. Patent Application Publication No. 2003-0004463.

When used in connection with thrombolytic pharmaceuticals, systems falling within the scope of the present invention can provide, for example, the benefit of integrated control and the ability to inject the thrombolytic pharmaceutical, to inject saline, and to periodically inject contrast to verify continued correct placement of the catheter.

Likewise, the systems of the present invention can be advantageously applied to tumor and other chemotherapy in which the chemotherapy pharmaceutical is supplied to the vessels supplying a tumor or other region of interest. In the case of chemotherapy pharmaceuticals, the fluid volumes can be quite small and an occlusion balloon can be beneficial to slow or prevent the wash out of the chemotherapy from, for example, tumor tissue.

The pharmaceuticals or drugs mentioned above, or other pharmaceuticals or drugs can be included in or associated with ultrasound bubbles. The systems of the present invention can deliver the bubbles to the region of interest and then ultrasound energy can be used to destroy the bubbles and promote the delivery of the drug to the tissue. The uses of ultrasound bubbles to deliver and release a pharmaceutical to a region of interest is disclosed in U.S. Pat. No. 6,397,098, assigned to the assignee of the present invention, the disclosure of which is incorporated herein by reference.

While procedures discussed herein in accordance with embodiments of the present invention have generally been described with respect to liquid drugs, it is to be understood that they can also apply to powdered drugs with either a liquid or gaseous vehicle, or gaseous drugs that are to be delivered to a recipient.

Without further analysis, the foregoing will so fully reveal the gist of the embodiments of the present invention that others can, by applying current knowledge, readily adapt it for various applications without omitting features that, from the standpoint of prior art, fairly constitute characteristics of the generic or specific aspects of the embodiments of the present invention.

If not otherwise stated herein, it may be assumed that all components and/or processes described heretofore may, if appropriate, be considered to be interchangeable with similar components and/or processes disclosed elsewhere in the specification, unless an express indication is made to the contrary.

If not otherwise stated herein, any and all patents, patent publications, articles and other printed publications discussed or mentioned herein are hereby incorporated by reference as if set forth in their entirety herein.

It should be appreciated that the apparatus and method of the present invention may be configured and conducted as appropriate for any context at hand. The embodiments described above are to be considered in all respects only as illustrative and not restrictive. All changes which come within the meaning and range of equivalency of the claims are to be embraced within their scope.

What is claimed is:

1. An arrangement for providing radiopharmaceutical, said arrangement comprising:
a carrying medium for carrying radiopharmaceutical to provide a radiation dose to a patient;
a first location at which radiopharmaceutical is provided to said transport medium;
a second location;
a first timing arrangement, associated with said first location, for ascertaining a first timepoint;
a second timing arrangement, associated with said second location, for ascertaining a second timepoint;
a first measuring arrangement, associated with said first location, for measuring a radiation dose;
an arrangement for ascertaining a radiation dose associated with said second location based on the radiation dose measured by said first measuring arrangement, the first timepoint and the second timepoint.

2. The arrangement according to claim 1, wherein said second location comprises a transport medium for transporting said carrying medium to a third location.

3. The arrangement according to claim 2, wherein said third location is associated with an arrangement for administering radiopharmaceutical to a patient.

4. A system for delivering an effective dose of a radiopharmaceutical material, comprising:
a source unit for dispensing the effective does from the radiopharmaceutical material;
a transport container for holding the effective dose;
a first timekeeping unit associated with the source unit, wherein the timekeeping unit obtains a first time data when the dose was dispensed from the source unit into the transportation container; and
a second timekeeping unit providing a second time when the effective dose is available for administration from the transport container;
wherein the second timekeeping unit is synchronized with the first timekeeping unit.

5. A system according to claim 4 wherein said data storage unit comprises an RFID tag.

6. A system according to claim 4 wherein said data storage unit comprises a bar code tag.

7. A system according to claim 4 wherein said data written to said data storage unit also comprises said radioactive decay half-life.

8. A system according to claim 4 wherein said data written to said data storage unit also comprises data that identifies said radiopharmaceutical material.

9. A system according to claim 4 wherein said first connection to said first timekeeping unit comprises a computer networking connection.

10. A system according to claim 4 wherein said second connection to said second timekeeping unit comprises a computer networking connection.

11. A system according to claim 4 wherein said time basis is provided by said first timekeeping unit.

12. A system according to claim 4 wherein said time basis is provided by a time basis standard.

13. A method for delivering an effective dose of a radiopharmaceutical material characterized by a radioactive decay half-life to a patient, comprising the steps of:
a. dispensing said radiopharmaceutical material from a source thereof into a radiopharmaceutical storage unit to which is affixed a data storage unit;
b. determining a first time when said radiopharmaceutical material was dispensed into said radiopharmaceutical storage unit using a first timekeeping unit;
c. measuring a radioactive activity of said radiopharmaceutical material at said first time with a measuring unit;
d. writing data onto said data storage unit, said data comprising said radioactive activity and said first time
e. placing said radiopharmaceutical storage unit into a patient dosing unit comprising a transfer unit to transfer said radiopharmaceutical material from said radiopharmaceutical storage unit into said patient;
f. placing said patient dosing unit proximate to said patient;
g. making a transference connection between said patient and said transfer unit;
h. reading said data from said data storage unit;
i. determining a second time when said effective dose is transferred into said patient using a second timekeeping unit, said second timekeeping unit being synchronized with said first timekeeping unit according to a time basis unit;
j. calculating said effective dose based on said first time, said second time, said radioactive activity, and said radioactive decay half-life; and
k. transferring said effective dose into said patient from said radiopharmaceutical storage unit by means of said transfer unit through said transference connection.

14. The method according to claim 13 wherein said data storage unit comprises an RFID tag.

15. The method according to claim 13 wherein said data storage unit comprises a bar code tag.

16. The method according to claim 13 wherein said data written to said data storage unit also comprises said radioactive decay half-life.

17. The method according to claim 13 wherein said data written to said data storage unit also comprises data that identifies said radiopharmaceutical material.

18. The method according to claim 13 wherein said time basis is provided by said first timekeeping unit.

19. The method according to claim 13 wherein said time basis is provided by a time basis standard.
